(19) United States
(12) Patent Application Publication
(10) Pub. No.: US 2018/0185397 A1
(43) Pub. Date: Jul. 5, 2018

(54) Isomalto-Oligosaccharide Prebiotic Formulations

(71) Applicant: Jamieson Laboratories Ltd., Windsor (CA)

(72) Inventor: John Doherty, Belle River (CA)

(21) Appl. No.: 15/861,696

(22) Filed: Jan. 4, 2018

Related U.S. Application Data
(60) Provisional application No. 62/562,358, filed on Sep. 22, 2017.
(60) Provisional application No. 62/442,408, filed on Jan. 4, 2017.

(51) Int. Cl. A61K 31/702; A61K 35/742; A61K 9/00; A61P 1/00; A23L 33/135; A23L 33/21

(52) U.S. Cl. CPC A61K 31/702; A61K 35/742; A61K 9/0056; A61K 2035/115; A23L 33/135; A23L 33/21; A61P 1/00

(57) ABSTRACT

The present invention relates to specific formulations of functional foods and nutraceuticals comprising probiotics and Isomalto-oligosaccharides in a tasty “gummy” dosage form. Embodiments of the invention provide relief from the unpleasant side effects of high fiber functional foods and nutraceuticals including stomach discomfort, flatulence, stomach rumbling/belching and overall gut feeling.

### RELATED APPLICATIONS

This application claims priority from U.S. provisional patent application Ser. No. 62/562,358, filed on Sep. 22, 2017, and U.S. provisional patent application Ser. No. 62/442,408, filed on Jan. 4, 2017,

### BACKGROUND OF THE INVENTION

Soluble fiber found in beans, oats, flaxseed and oat bran may help lower total blood cholesterol levels by lowering low-density lipoprotein, or “bad,” cholesterol levels. Studies have shown that high-fiber foods may have other heart-health benefits, such as reducing blood pressure and inflammation. Isomalto-oligosaccharide (“IMO”) is a naturally occurring, plant-based dietary fiber. It is prebiotic, soluble, and low-glycemic. Fermentation of IMO by colonic bacteria results in production of Short Chain Fatty Acids (SCFA) that metabolize in liver and are thought to confer many physiological benefits to the host including: protection from colonic cancer, stabilize blood glucose levels, decrease cholesterol synthesis, lowering the digestive tracts pH which allows for an increase absorption of dietary minerals and help stimulate the immune function. See http://www.albertsons.com/isomalto-oligosaccharide-the-natural-prebiotic-plant-fiber-explained/accessed Dec. 20, 2016.

Prebiotics are substances that induce the growth or activity of microorganisms that may contribute to the well-being of their host. One example where prebiotics may help is in the gastrointestinal tract, where prebiotics can alter the composition of organisms in the gut microbiome. Prebiotics are generally non-digestible fiber ingredients that pass undigested through the upper part of the gastrointestinal tract and stimulate the growth or activity of advantageous bacteria that colonize the large bowel by acting as substrate for them. Hutkins R W, Krumbeck J A, Bindels L B, Cani P D, Fahey G Jr., Goh Y J, Hamaker B, Martens E C, Mills D A, Rastal R A, Vaughan E, Sanders M E (2016). “Prebiotics: why definitions matter”. Curr Opin Biotechnol. 37: 1-7.

Isomalto-oligosaccharides are mixed linkage oligosaccharides, having mixtures of 1,4 alpha and/or 1,6 alpha glucosidic linkages. IMOs contain a substantial amount of branched oligo-saccharides such as isomaltose, panose, isomaltotriose, isomaltotetraose, isopanose and higher branched oligo-saccharides. See U.S. Pat. Nos. 7,993,689 and 6,025,168.

Isomalto-oligosaccharide is known to be safe up to 15 g/serving and up to 30 g/day without digestive discomfort.

Examples of IMO products include: seasonings (mayonnaise, vinegar, soup base etc.), confectionery (candy, chewing gum, chocolate, ice cream, sherbet, syrup), processed foods of fruits and vegetables (jam, marmalade, fruit sauces, pickles), meat or fish foods (ham, sausage, etc.), bakery products (bread, cake, cookie, pastry), precooked foods (salad, boiled beans, etc.), canned and bottled foods, convenience foods (instant coffee, instant cake base, etc.), and beverages, both alcoholic (liquor, seju, wine, sake, beer [International Patent Publication No. WO 02/20712 A1], etc.) and non-alcoholic (coffee, juice, nectar, aerated or carbonated drinks, lemonade, cola). Isomalto-oligosaccharide can further be applied as ingredients in animal feed and pet foods. Non-food application areas are cosmetics and medicine (cigarette, lipstick, toothpaste, internal medicine, etc.) see U.S. Pat. No. 7,993,689.

Isomalto-oligosaccharides belong to a group of oligosaccharides classified as functional-health food oligosaccharides and include fructo-oligosaccharides, galacto-oligosaccharides, xylo-oligosaccharides and gentio-oligosaccharides. IMO's have been linked to the increase in general well being when taken orally on a regular daily basis and are called “probiotics”. Prebiotics are defined as non-digestible substances that have some biological effect by stimulation of growth or bioactivity of beneficial microorganisms in the intestine. (Przemyslaw Jan Tomasik and Piotr Tomasik. 2003 American Association of Cereal Chemists, Inc. 80(2): 113-117).

Oligosaccharides increase the number of bifidobacteria and lactobacilli in the large intestine and reduce the concentration of putrifactive bacteria. Bifidobacteria support some health promoting properties like the inhibition of the growth of pathogens. They are also associated with such diverse effects as the modulation of the immune system, the reduction of the levels of triglycerides and cholesterol, the production of vitamins (B group), the reduction of blood ammonia concentrations, the prevention of translocation, the restoration of the normal gut flora after anti-microbial therapy, the production of digestive enzymes, the reduction of antibiotic associated side effects (Kohmoto T., Fukui F., Takaku H., Machida Y., et al., Bifidobacteria Microflora, 7(2)(1988), 61-69; Kohmoto K., Tsuji K., Kaneko T. Shiota M., et al., Biosc. Biotech. Biochem., 56(6)(1992), 937-940; Kaneko T, Kohmoto T., Kikuchi H., Fukui F., et al., Nippon Nogeikagaku Kaishi, 66(8)(1992), 1211-1220, Park J-H, Jin-Young Y., Ok-Ho S., Hyun-Kyung S., et al., Kor. J. Appl. Microbiol. Biotechnol., 20(3)(1992), 237-242).

The isomalto-oligosaccharides are synthesized by a transglucosylation reaction using a D-glucosyltransferase. As a result of transglucosidase reactions, the malto-oligosaccharides are converted into isomalto-oligosaccharides resulting in oligosaccharides having a higher proportion of glucose moieties linked by alpha-D-1,6 glucosidic linkages. (McCleary B. V., Gibson T. S., Carbohydrate Research 185(1989)147-162; Benson C. P., Kelly C. T., Fogarty W. M., J. Chem. Tech. Biotechnol., 32(1982)790-798; Pazur J. H., Tominaga Y., DeBrosse C. W., Jackman L. M., Carbohydrate Research, 61(1978) 279-290).

Isomalto-oligosaccharides can be obtained in different ways. For example, glucose syrups at high dry solids concentration i.e. 60-80% are treated with glucoamylase resulting in the formation of isomalto-oligosaccharides. Other examples are maltose transfer achieved by addition of pullulanase to liquefied starch, branching of maltose syrups and treatment of sucrose with dextran sucrase. See U.S. Pat. No. 6,025,168.

### Probiotics

As defined by an expert panel convened in 2002 by the Food and Agriculture Organization of the United Nations (FAO) and the World Health Organization (WHO), probiotics are “live microorganisms administered in adequate amounts that confer a beneficial health effect on the host” (FAO/WHO, 2002).

Probiotics are often called “good” or “helpful” bacteria because they help keep your gut healthy. Probiotics are naturally found in your body. You can also find them in some foods and supplements. Probiotics are thought to work by helping to balance your “good” and “bad” bacteria to keep your body working like it should.

Many types of bacteria are classified as probiotics. They all have different benefits, but most come from two groups: 1) Lactobacillus; and 2) Bifidobacterium. Lactobacillus are the most common probiotic. Lactobacillus are found in yogurt and other fermented foods. Different strains can help with diarrhea and may help digest lactose, the sugar in milk. Bifidobacterium are found in some milk products and are used to ease the symptoms of irritable bowel syndrome (IBS), among other diseases. Mary Jo DiLonardo, WebMd http://www.webmd.com/digestive-disorders/features/what-are-probiotics#1 accessed Nov. 1, 2016.

There is encouraging evidence that probiotics may help:
Treat diarrhea, especially following treatment with certain antibiotics
Prevent and treat vaginal yeast infections and urinary tract infections
Treat irritable bowel syndrome
Speed treatment of certain intestinal infections
Prevent or reduce the severity of colds and flu

“Do I need to include probiotics and prebiotics in my diet?” Answers from Katherine Zeratsky, R.D., L.D. at http://www.mayoclinic.orehealthy-lifestyle/consumer-health/expert-answers/probiotics/faq-20058065 accessed Nov. 1, 2016.

In over 50 years, Bacillus species have been used as probiotics and in recent years the scientific interest in Bacillus species as probiotics has significantly increased (Mazza, 1994; Sanders et al., 2003; Hong et al., 2005; Cutting, 2011).

Gummies are gelatin-based chewable candy that have a long history as a popular confectionary, first introduced in 1920 as gummi bears. Currently, gummies are available in a wide variety of shapes, sizes and flavors. The primary ingredients include water, gelatin, sweeteners, flavors, and colors.

The main ingredient responsible for the gummy candy's unique, gummy characteristics is gelatin. This is a protein derived from animal tissue that forms thick solutions or gels when placed in water. When used at an appropriate concentration, the gels take on the texture of the chewy, gummy candy. However, since these gels are thermoreversible, which means they get thinner as they are heated, gummy candies have a “melt in the mouth” characteristic. Both the texture and the amount of time it takes the candy to dissolve in the mouth can be controlled by the amount of gelatin used in a recipe. See http://www.madehow.com/Volume-3/Gummy-Candy.html#ixzz4Pjh835zT accessed Dec. 19, 2016; and P. Delgado & S. Bañón (2015) Determining the minimum drying time of gummy confections based on their mechanical properties, CyTA—Journal of Food, 13:3, 329-335, DOI: 10.1080/19476337.2014.974676

Recently gummies have become a popular dosage form for dietary supplements such as vitamins, especially for children or others who don't like to swallow other oral dosage forms.

Abandoned U.S. patent application Ser. No. 12/547,355 discloses chewable compositions, formulas and methods for manufacturing and delivering pharmaceutical and supplement compounds. The specific formulations and methods of the instant application with all of the attendant surprising advantages are not disclosed in this application.

Expired U.S. Pat. No. 9,022,244 discloses consumable, gummy delivery systems and methods of making them. The gummy delivery systems include an active ingredient mixed with a glycerylated gelatin matrix prepared by heating an aqueous solution of gelatin and glycerin to a temperature and for a time sufficient to remove some of the moisture content of the initial aqueous solution. The active ingredient can be delivered from a shearform matrix carrier. The specific formulations and methods of the instant application with all of the attendant surprising advantages are not disclosed in this application.

Patent application publication WO2006007470A1 discloses food products comprising a food base and the chocolate or cocoa butter encapsulated pro-biotic, especially lactic acid forming cultures, as a coating or portion or phase of the food product.

US Patent application 20120015075 discloses a chewable composition for the oral delivery of live microorganisms. The chewable composition includes a delivery vehicle and an active ingredient incorporated therein. The delivery vehicle may include an organic or non-organic gummy candy including a binding agent, sweetener, flavoring, and/or coloring. The active ingredient may include a predetermined amount of at least one probiotic. The delivery vehicle may also include a predetermined amount of at least one prebiotic. The delivery vehicle may also include any combination of nutraceuticals, vitamins, minerals, antioxidants, soluble and insoluble fiber, herbs, plants, probiotics, probiotics, antioxidants, amino acids, fatty acids, digestive enzymes, dietary supplements, or any other health promoting ingredient. The specific formulations and methods of the instant application with all of the attendant surprising advantages are not disclosed in this application.

All of the foregoing publications are all incorporated herein by reference in their entirety, and for the teachings contained therein.

### DETAILED INVENTION DISCLOSURE

The present invention relates to specific formulations and methods related to functional foods and nutraceuticals comprising probiotics and Isomalto-oligosaccharides in a tasty “gummy” dosage form (referred to herein as “IMO/PRO Gummies”). Embodiments of the invention provide relief from the unpleasant side effects of high fiber functional foods and nutraceuticals including stomach discomfort, flatulence, stomach rumbling/belching and overall gut feeling.

The isomalto-oligosaccharide (IMO) mixture according to the invention consists of 2 to 9 glucose units linked together by digestion-resistant α(1,6) linkages, providing dietary fiber and prebiotic functionalities as well as having a relative sweetness equal to 50-60% of sucrose. See U.S. Pat. Nos. 7,582,453 and 7,906,314 as well as Canadian Patent Nos. 2475817 and 2474999. The enzymatic manufacturing process controls the degree of polymerization and the α(1,6) linkages to ensure consistent quality of isomalto-oligosaccharide. It is a low calorie sweetener with a dietary fiber functionality to help in human digestive health.

The following information is extracted from the FDA's GRAS Notice Archive No. 526: Bacillus coagulans strain Unique IS2 spores preparation Mar. 23, 2015. http://www.accessdata.fda.gov/scripts/fdcc/index.cfm?set=GRASNotices&id=526 accessed Dec. 19, 2016 incorporated by references in its entirety herein.

Bacillus species have been used as probiotics and in recent years and the scientific interest in Bacillus species as probiotics has significantly increased (Mazza, 1994; Sanders et al., 2003; Hong et al., 2005; Cutting, 2011). These species are spore forming bacteria that are commonly found in nature. Bacillus spores are stable at high temperatures and in acidic conditions. Compared to non-spore formers, such as Lactobacillus species, spores provide advantages as the product can be stored at room temperature in a desiccated form without any deleterious effect on viability; spores are capable of surviving the low pH of the gastric barrier (Barbosa et al., 2005; Spinosa et al., 2000; Tuohy et al., 2007); and, the entire dose of ingested bacteria can reach the small intestine intact and germinate. Additionally, it can survive industrial food manufacturing conditions and ensures long term viability (Sanders et al., 2001).

Bacillus coagulans is a lactic-acid producing species that has typical characteristics of both Lactobacillus and Bacillus genera. It was first isolated and described in 1932 by Horowitz and Wlassowa. At the time it was named Lactobacillus sporogenes (Bergey et al., 1939). This microorganism possesses a protective, spore-like protein coating (endospores) that protects it from the acidic conditions in the stomach. Thus, it can reach the small intestine, germinate, and multiply. It is also an economically important species, frequently used in the production of optically pure lactic acid, coagulin, and other thermostable enzymes (Su et al., 2012).

B. coagulans has been marketed as a probiotic to maintain the ecological balance of the intestinal microflora and normal gut function.

B. coagulans and B. subtilis are marketed as dietary supplement probiotics for human consumption (Sanders et al., 2003; Hong et al., 2008). B. coagulans has been sold as a dietary supplement under different names such as Nature's Plus, GanedenBC3°, Pit-Stop, Fresh Start Bolus, GutFlora (VSL-3), Tarm-X Balans™, Sanvita, Ampilac, Sprolac®, Bactlyte, etc. In addition to this, B. coagulans is also marketed as a constituent of other dietary supplements. In products like these, B. coagulans is formulated with other ingredients, including bifidobacteria, lactobacilli, minerals, vitamins (particularly B complex), or prebiotics. The recommended dose for B. coagulans ranges from 3.6×108 to 1.5×109 cfu/capsule, two or three times per day for a healthy adult.

In addition to its use as a dietary supplement B. coagulans is used in conventional foods (Ganedan, 2011). B. coagulans is used in a variety of foods at levels up to approximately 2×10 9cfu/serving. The acceptable daily intake (ADI) is 93.8×10 cfu/person/day. The US FDA has approved the use of B. coagulans in the production of enzymes that can be used in the manufacturing of foods. B. coagulans (a nonpathogenic and nontoxicogenic microorganism) is also recognized as GRAS (21 CFR 184.1372) in the production of insoluble glucose isomerase enzyme.

Similar to the US FDA, the Health Canada has permitted the use of B. coagulans in the production of glucose isomerase enzyme. Furthermore, the FDA's Center for Veterinary Medicine has also recognized the use of B. coagulans as GRAS for veterinary purposes.

The European Food Safety Authority (EFSA) has granted Qualified Presumption of Safety (QPS) status of B. coagulans, since 2008 (EFSA, 2012). This suggests that B. coagulans can be safely used in European countries.

The Japanese Ministry of Health and Welfare has approved the use of B. coagulans products for improvement in symptoms caused by abnormalities in the intestinal flora or in dysbiosis (Majeed and Prakash, 1998).

The available information suggest that B. coagulans has been used as part of the fermenting process for the production of a protein-rich food known as “ugba” that is commonly consumed in Nigeria (Isu and Njoku, 1997).

The role of lactic acid bacteria has been extensively studied in the intestinal microecology. These bacteria play an important role in maintaining the healthy digestive tract (Adams, 1999; Soomro et al., 2002; Ouwehand et al., 2004). Among the different lactic acid producing bacteria, B. coagulans has a long history of use for its potential health benefits. Sanders et al. (2003) reviewed several Bacillus species for their potential probiotic characteristics. These investigators noted that B. coagulans has been evaluated for probiotic functionality and sold worldwide for both human and animal uses. In another article, Catanzaro and Green (1997) considered B. coagulans as beneficial bacteria. This microorganism was first isolated in 1932 (Horowitz-Wlassowa and Nowotelnow, 1932). The potential gastrointestinal benefits of B. coagulans and other spore-forming bacteria have been investigated during 1958 and 1959 (Guida et al., 1958; Guida and Guida, 1959). The available information indicate that B. coagulans has been in use for over five decades.

B. coagulans Unique IS2 is a gram-positive, catalase-positive, spore forming, rod-shaped, slightly acidophilic, thermotolerant, aerobic to microaerophilic, highly resilient bacteria.

B. coagulans Unique IS2 is manufactured through a specific time and temperature controlled fermentation of suitable ingredients. The raw material mixture for fermentation is sterilized, cooled and inoculated with B. coagulans Unique IS-2. The inoculum is allowed to incubate to the fermentation endpoint under constant temperature and aeration. After the required incubation period, the biomass is collected by centrifugation. Subsequently, the concentrated suspension is dried by spray drying. The dried culture is then placed and stored in a dry condition environment at room temperature. The processing aids, fermentation medium and diluents used in the manufacturing of B. coagulans Unique IS2 are either approved as food additives or are GRAS substances.

The finished B. coagulans Unique IS2 product is prepared from an approved concentrated product, by diluting with food grade diluents such as maltodextrin and/or microcrystalline cellulose powder (MCCP) and/or fructooligosaccaharides (FOS). These diluents are recognized as safe for the intended uses. Use of maltodextrin is GRAS as per 21 CFR 184.1444.

The practice of certain embodiments of the invention is illustrated in the following non-limiting examples. Having read the present specification and claims, one skilled in the art it will readily appreciate that many variations, changes, modifications and additions to the methods and compositions described herein are possible without deviating from the broader invention that is disclosed and taught herein. Such variations, changes, modifications and additions are within the scope of the present invention.

### EXAMPLES

### Example 1: Imo/Pro Gummy Manufacturing

A method for manufacturing a delivery system of the present invention is disclosed. In certain embodiments, the method of manufacturing involves three main phases: (i) mixing and storing; (ii) batching and cooking; and (iii) depositing and curing.

The first phase of mixing and storing begins with water and a binding agent are mixed in a mixing tank to form a gelling compound. In one embodiment, the mixing tank may include a 1,000 gallon stainless steel planetary mixer, a scrape surface mixer, a holding tank with an agitator, or any other suitable mixer. During production, water and the binding agent are continuously mixed in the mixing tank and the gelling compound is continuously turned in the tank by an agitator to keep the binding agent suspended in water (i.e., to prevent the binding agent from settling on the bottom of the mixing tank).

The gelling compound may include cold, warm, or hot water. However, warm or hot water may be used to reduce the hydration time (i.e., the time it takes the water to hydrate the binding agent) of the gelling compound. For example, about 250 lbs of gelatin mixed with about 250 lbs of warm water may reach a homogenous mixture in about 10 minutes. The hydration rate of the gelling compound may also vary according to the speed of the agitator.

The binding agent may include a pectin gel, gelatin, food starch, or any combination thereof. Depending on the binding agent used, the gelling compound may include, for example only, one of the following formulations:

In one implementation, a buffer, such as sodium bisulfate or sodium citrate, may be mixed into the gelling compound to regulate the pH of the mixture. In one implementation, the gelling compound may contain approximately 0.01 to 0.03% buffer by weight, or any other suitable amount. The pH of the mixing tank may be adjusted to a range from about 3.2 to about 4.5 to provide adequate gelation and to ensure that the gelling compound does not become unstable (or acidic) during mixing.

At this step the gelling compound may be filtered through a fine mesh, to remove particulates in the slurry, and stored in a holding tank. In one implementation, about 140 lbs to 190 lbs of gelling compound may be delivered from the mixing tank to the holding tank every 5 to 10 minutes. The filter may be a 0.034 inch stainless steel basket strainer and the holding tank may be a 1,500 gallon stainless steel tank. In some implementations, the holding tank may include a moderate agitator (e.g., mixing blades) to mix the compound and prevent the binding agent from settling on the bottom of the holding tank during storage.

From the holding tank, approximately 125 lbs to 185 lbs of gelling compound may be delivered to a mixing vessel every 5 to 10 minutes, for example. In one implementation, the mixing vessel may be a 5,000 gallon stainless steel planetary mixer. In other implementations, the mixing vessel may be a scrape surface mixer, a holding tank with an agitator, or any other type of suitable mixer.

In the mixing vessel, water, additives, supplements, and an active ingredient may be added to the gelling compound to form a sugar slurry. In one implementation, the additives may include sodium citrate, sweeteners such as sugar (also referred to herein as sucrose or natural cane juice) and/or syrup (e.g., corn, glucose, rice, tapioca), and corn starch, in liquid and/or powdered form. In one implementation, the supplements may include vitamins, minerals, herbs, plants, amino acids, enzymes or any other supplements digested to promote the heath and well-being of a person.

### Example 2: The Automated Imo/Pro Gummy Manufacturing Process

In certain embodiments, Imo/Pro gummy manufacturing uses a starch molding process on an automated Mogul machine. First the gummy slurry is made, and then it is deposited into starch lined trays. The filled trays are cooled and the resulting formed gummy is emptied from the trays for final coating and packaging. This process may be a continuous cycle which allows for recycling of major componentry. The process may be broken down into 3 main stages.

### Stage 1—Compounding

I. Physically pour the appropriate amount of gummy raw material consisting of water, pectin and/or gelatin, and a supplement blend into a main mixing vessel.
II. For gelatin based gummies, the gelatin is first mixed with hot water at 80° C. for about 20 minutes, followed by the addition of the pectin and supplement blend, which is mixed until completely dissolved. For pectin based gummies, the pectin and supplement blend are mixed in water until completely dissolved.
III. After mixing and dissolution, the blend is transferred to a cooker and heated to 130° C.
IV. The blend is then transferred to the color flavor acid deck where the heat sensitive ingredients (citric acid, natural color and natural flavor) are added to the mixture system at 80° C. Depending on the size of the batch, the gummy slurry can take from one to three hours to be compounded. After this stage the final Compounded Slurry is ready for forming.

### Stage 2—Filling, Forming and Coating

I. The forming stage of the gummies is completed in a Mogul machine. Compounded Slurry from Stage 1 steps I-IV, is transferred to the Mogul depositors. The depositors have a line of filling nozzles for delivery of the exact amount of gummy slurry needed per unit into the trays as they pass under it. The depositor section of the Mogul may contain 30 or more depositors, depending on how many imprints there are on the trays lined with starch. The filled trays from the depositors are moved along to a stacking machine and then sent to a cooling room, where they stay until they are appropriately cooled and formed. This part of the process can take over 24 hours depending on base and ingredient combination.
II. Trays that contain previously filled, cooled, and formed gummies are stacked. The trays are then removed from the stack one-by-one along a conveyor belt into the next section of the machine, known as the starch buck.
III. As they enter the starch buck, the trays are inverted and the gummies fall out into a vibrating metal screen known as a sieve. The vibrating action of the sieve, in concert with oscillating brushes, removes all of the excess starch that adheres to the gummies. These pieces then move along a conveyor belt to trays. In certain embodiments, a pneumatic starch buck may be used to further automates this step. In this device, a tightly fitting cover is placed over the filled trays. When it is inverted, the gummies adhere to the cover and remain in their ordered position. The excess starch is then removed by fast-rotating compressed-air jets.
IV. Finally, the gummies are oil coated or dusted using a rotary drum to avoid a sticky surface, passed through an inspection belt and metal detector to ensure high quality before packaging. The completed gummies are collected for final packaging, the trays are moved back to the Mogul for recycling.

### Stage 3—Starch Molding and Recycling

I. A starch molding machine can automated many aspects of the manufacturing process. In this machine, starch has three primary purposes:
   1. First, it prevents the gummies from sticking to the molds, which allows for easy removal and handling.
   2. Second, it holds the gummy in place during the drying, cooling, and setting processes.
   3. Finally, it absorbs moisture from the gummies, giving them the proper texture.
II. The starch that is removed from the gummies may be reused in the process, but first it must be cleaned, dried, and otherwise reconditioned. Gummy particles are first removed by passing the starch through a metal screen known as a sieve. It is then conveyed to a recirculating starch conditioning system.
III. As it enters this machine, it is dried by being passed through hot, moving air. After drying, the starch is cooled by cool air jets and conveyed back out to the Mogul to be reused in the starch molding process.
IV. The starch returns from the dryer via a conveyor belt to the Mogul, where it is filled into the empty trays and leveled. These were the same trays that were inverted and emptied in stage 2 step III. These starch-filled trays then move to a printer table. Here, a board that has the inverse of the mold printed on it presses the starch down so the mold has an indent in it. From here, the trays are moved to the depositors as the recycling is completed.

### Example 3: IMO/PRO Gummy Formulas

Each serving (4 gummies) of IMO/PRO Gummy includes a 2.25 g serving of fiber as Isomaltooligosaccharide derived from Zea mays plus a probiotic dose of 250 million CFU Bacillus coagulans IS-2 encapsulated in a great tasting gummy dosage form made of pectin, citric acid, sodium citrate, natural flavors (such as mixed berry), natural colors (for example purple carrot juice and fruit juice), corn starch, beeswax, coconut oil, Brazilian palm tree wax, monk fruit extract. Table 1 provides details of the ingredients according to the present invention other than the probiotic.

### Table 1: Ingredients

TABLE 1
Ingredients

| Ingredients | |
|---|---|
| (listed in descending order) | Source Material |
| Fiber syrup | Corn Starch (non-GMO) |
| Fiber powder | Corn Starch (non-GMO) |
| Pectin | Peel of citrus spp. |
| Citric acid | Fermentation of corn syrup |
| Natural flavors | Natural flavor |
| Black carrot extract | Daucus carota |
| (natural color) | |
| Sodium citrate | Reaction of fermentation derived citric |
| | acid with sodium carbonate |
| Natural fruit juice color | Fruit juice concentrated |
| blend (blue) | |
| Fruit sweetness | Monk fruit extract |
| Vegetable oil/Coconut oil | Fractioned, refined, bleached, |
| | deodorized, (non-hydrogenated) |
| | oil of Cocos nucifera |
| Beeswax | Wax of Apis mellifera |
| Starch | Corn derived from rhizomes |

### Example 4. Gummy Clinical Trial

The objective of this trial was to assess the measurable physiological effects between two separate gummy fiber formulations. The fiber comparison is an embodiment of the instant invention comprising Isomaltooligosaccharide (IMO) versus Inulin, both of which are soluble fibers.

Isomaltooligosaccharide alone has been studied however, however, the combination of IMO fibre and probiotics according to the present invention has yet to be explored; therefore this trial investigated this novel combination of actives according to the invention by evaluating the measurable dietary effects of IMO fibre and probiotics according to the present invention in comparison to an Inulin control. Key markers assessed were the recall of the following GI symptoms associated with high fiber intake but not limited to; flatulence, stomach rumbling, increased bowel movement frequency, consistency, and/or stomach discomfort.

### Materials & Methods

Healthy individuals (n=31) were recruited for the study. Exclusion criteria involved individuals taking or during course of trial prescribed anti-biotics, diagnosed GI disorders and/or subjects with related disorders affecting GI tract. A high dropout rate was estimated due to the length (3 period design) of the study.

The study was a randomized, double-blind, three period crossover study, participants were provided with a week supply of both an embodiment of the invention (IMO+Probiotics gummies) and an active control (Inulin) prepared in a similar dosage form. Participants were told to consume either treatment for a period of 1 week with a 1 week washout before switching to alternate treatment for 1 week. Doubling effect of the crossover design increased the power of the analytical results and allowed for the possible loss of participants by voluntary dropout or illness.

Each gummy of the embodiment of the Invention included a 2.25 g serving of fiber as Isomaltooligosaccharide derived from Zea mays plus a probiotic dose of 250 million CFU Bacillus coagulans IS-2. Participants were told to chew four (4) gummies in the morning daily.

Each gummy of Control included 2 g of Inulin fiber from Chicory root. Participants were told to chew two (2) gummies in the morning daily. The lesser amount of fiber in the control was used to further prove the beneficial tolerability results of the instant invention.

Participants were asked to return gummy sample bags anonymously at the end of one week treatment period to assess compliance to the dosing protocol.

Treatment began with Invention Embodiment for 1 week. A 1 week washout period then followed. The study concluded with a one week treatment of Control. In total, the study ran over 3 weeks.

Before and after treatment participants were asked to complete the overall GI Questionnaire using a VAS (Visual Analogue Scale) on the following measurements which allowed for analysis of the intended physiological effects as well as the possible side effects with such key ingredients. These measurements included stomach discomfort, flatulence, stomach rumbling/belching and overall gut feeling. In addition, participants were also asked to fill out the daily testimonial page, where they recorded the daily number of soft/loose, typical and/or firm bowel movements.

A 1 week wash out period then followed. The process then began again with the second treatment.

The intent of the data collected was to compare the results of both interventions to see which had the most positive effects with the least negative/unintended effects. The intent of daily recall was to see if there was an effect change (adjustment period) over the course of the treatment duration.

A total of 19 participants returned the study booklets (1 participant did not begin the study due to personal reasons). As expected a high dropout rate occurred with 11 participants not returning the study booklets most likely due to the length of the study and compliance required. Of the 18 individuals that returned booklets, 3 discontinued treatment with Control due to unwanted side effects of gas, bloating and abdominal discomfort. All 18 individuals completed the full treatment of Invention Embodiment.

Each individual scored their GI symptoms on a scale of 0-20, with participants classified into one of three tiers (0-10: Overall Gut Feeling is Upset to Regular/10: Overall Gut Feeling is Regular/10-20: Overall Gut Feeling is Regular to Great).

A total of 16/18 participants (89%) taking Invention Embodiment had a GI Questionnaire score between 10-20 at baseline and after treatment. The two individuals who had a score of 0-10 after treatment, also had a similar score (1 point difference) at baseline treatment, and therefore experienced abdominal discomfort before treatment. For the majority of individuals, surprisingly, no significant differences (drop of more than 2 points) were found between baseline and after treatment for the embodiment of the invention (one outlier had a drop of 4 points still falling within the 10-20 score range) (see Table 2).

TABLE 2
Invention Embodiment (IMO & Probiotic) GI Questionnaire
Before and After Scores
PRODUCT K (IMO & Probiotic) GI QUESTIONNAIRE

| | Total Score | Total Score |
|---|---|---|
| | (Before Treatment) | (After Treatment) |
| Participant 1 | 16 | 16 |
| Participant 2 | 16 | 16 |
| Participant 3 | 17 | 17 |
| Participant 4 | 16 | 16 |
| Participant 5 | 17 | 16 |
| Participant 6 | 15 | 16 |
| Participant 7 | 17 | 16 |
| Participant 8 | 17 | 19 |
| Participant 9 | 18 | 14 |
| Participant 10 | 10 | 10 |
| Participant 11 | 12 | 11 |
| Participant 12 | 13 | 14 |
| Participant 13 | 10 | 10 |
| Participant 14 | 13 | 13 |
| Participant 15 | 14 | 15 |
| Participant 16 | 4 | 5 |
| Participant 17 | 11 | 13 |
| Participant 18 | 7 | 8 |

A total of 16/18 (89%) participants had a baseline score of 10-20. However, after treatment a total of 12/18 participants (67%) taking Control had a GI Questionnaire score which fell within the 0-10 range, with only 6/18 remaining in their baseline tier. A majority of participants complained of gas, bloating and abdominal discomfort, with 3 individuals discontinuing the study due to excessive adverse dietary side effects (see Table 3).

TABLE 3
Control (Inulin) GI Questionnaire Before and After Scores
PRODUCT K (IMO & PROBIOTIC) GI QUESTIONNAIRE

| | Total Score | Total Score |
|---|---|---|
| | (Before Treatment) | (After Treatment) |
| Participant 1 | 16 | 9 |
| Participant 2 | 16 | 18 |
| Participant 3 | 17 | 5 |
| Participant 4 | 16 | 4 |
| Participant 5 | 17 | 4 |
| Participant 6 | 15 | 13 |
| Participant 7 | 17 | 4 |
| Participant 8 | 17 | 5 |
| Participant 9 | 18 | 5 |
| Participant 10 | 10 | 8 |
| Participant 11 | 12 | 11 |
| Participant 12 | 13 | 7 |
| Participant 13 | 10 | 13 |
| Participant 14 | 13 | 6 |
| Participant 15 | 14 | 20 |
| Participant 16 | 4 | 4 |
| Participant 17 | 11 | 13 |
| Participant 18 | 7 | 10 |

An average GI Questionnaire score of 14 was found at baseline and after treatment for IMO Fibre according to the Invention. However, an average GI Questionnaire score of 9 for the Inulin control was found after treatment signifying a significant drop in comparison to Inulin baseline, and in comparison to IMO treatment (see Table 4).

TABLE 4
Comparing Mean Scores of Invention Embodiment (IMO and Probiotic
Fibre Gummy) & Control (Inulin Gummy) GI Questionnaire
COMPARISON OF MEAN SCORES FOR PRODUCT K (IMO and
Probiotic Fibre Gummy) & PRODUCT H (Inulin Gummy)
GI QUESTIONNAIRE

| | Mean Score | Mean Score |
|---|---|---|
| | (Before Treatment) | (After Treatment) |
| PRODUCT K | 14 | 14 |
| (IMO & Probiotic) | | |
| PRODUCT H | 14 | 9 |
| (Inulin Fibre) | | |

More inconsistencies of bowel movements were noted in participants taking Control in contrast to Invention Embodiment. There was a wide range of stool descriptions listed for both treatments with no significant differences or patterns observed at baseline and after treatment (see Tables 5 and 6).

TABLE 5
Bowel Movements For Invention Embodiment
(IMO and Probiotic Fibre Gummy)
Product K (IMO & Probiotic) Bowel
Movements Tracked Over 7 Day Treament

| | Base- | Day | Day | Day | Day | Day | Day | Day |
|---|---|---|---|---|---|---|---|---|
| | line | One | Two | Three | Four | Five | Six | Seven |
| Participant 1 | 2 | 2 | 2 | 3 | 2 | 2 | 3 | 2 |
| Participant 2 | 1 | 1 | 2 | 3 | 2 | 2 | 2 | 1 |
| Participant 3 | 1 | 1 | 1 | 1 | 1 | 1 | 1 | 1 |
| Participant 4 | 1 | 1 | 1 | 1 | 1 | 1 | 1 | 1 |
| Participant 5 | 1 | 3 | 3 | 2 | 1 | 1 | 1 | 1 |
| Participant 6 | 1 | 1 | 1 | 1 | 1 | 1 | 1 | 1 |
| Participant 7 | 1 | 1 | 1 | 1 | 1 | 1 | 1 | 1 |
| Participant 8 | 2 | 2 | 1 | 2 | 1 | 1 | 2 | 2 |
| Participant 9 | 2 | 2 | 2 | 2 | 1 | 2 | 1 | 2 |
| Participant | 2 | 2 | 2 | 1 | 2 | 2 | 1 | 1 |
| 10 | | | | | | | | |
| Participant | 2 | 1 | 2 | | 1 | 1 | 2 | 2 |
| 11 | | | | | | | | |
| Participant | 1 | 1 | 1 | 1 | 1 | 1 | 1 | 1 |
| 12 | | | | | | | | |
| Participant | 2 | 2 | 1 | 1 | 1 | 1 | 2 | 2 |
| 13 | | | | | | | | |
| Participant | 1 | 1 | 1 | | 1 | 1 | 1 | 1 |
| 14 | | | | | | | | |
| Participant | 1 | 3 | 1 | 1 | 1 | 1 | 2 | |
| 15 | | | | | | | | |
| Participant | 2 | | 1 | 1 | 1 | 1 | 1 | 1 |
| 16 | | | | | | | | |
| Participant | 2 | 2 | 2 | 1 | 2 | 2 | 3 | 2 |
| 17 | | | | | | | | |
| Participant | 3 | 3 | 3 | 3 | 3 | 2 | 2 | 2 |
| 18 | | | | | | | | |

TABLE 6
Bowel Movements For Control Control (Inulin Gummy)
PRODUCT H (Inulin Fibre) Bowel
Movements Tracked Over 7 Day Treament

| | Base- | Day | Day | Day | Day | Day | Day | Day |
|---|---|---|---|---|---|---|---|---|
| | line | One | Two | Three | Four | Five | Six | Seven |
| Participant 1 | 2 | 2 | 4 | 3 | 4 | 2 | 4 | 2 |
| Participant 2 | 1 | 3 | 3 | 3 | 2 | 2 | 2 | 2 |
| Participant 3 | 1 | 1 | 1 | 1 | 1 | 1 | 1 | 1 |
| Participant 4 | 1 | 1 | 1 | 1 | 1 | 1 | 1 | 1 |
| Participant 5 | 1 | 1 | 1 | 2 | 2 | 2 | 3 | 3 |
| Participant 6 | 1 | 1 | 1 | 1 | 1 | 1 | 1 | 1 |
| Participant 7 | 1 | | 1 | 1 | | 2 | | 1 |
| Participant 8 | 2 | 1 | 2 | 2 | 1 | 2 | 3 | 2 |
| Participant 9 | 2 | 1 | 5 | 4 | | | | |
| Participant | 2 | 2 | 3 | 2 | 2 | 2 | 1 | 3 |
| 10 | | | | | | | | |
| Participant | 2 | 2 | 1 | 2 | 1 | 2 | 1 | |
| 11 | | | | | | | | |
| Participant | 1 | | 1 | 2 | 2 | | | |
| 12 | | | | | | | | |
| Participant | 2 | 2 | 2 | 1 | 1 | 1 | 2 | 1 |
| 13 | | | | | | | | |
| Participant | 1 | | 1 | 1 | 2 | 1 | 1 | 1 |
| 14 | | | | | | | | |
| Participant | 1 | 2 | 1 | 1 | 2 | 2 | 2 | 2 |
| 15 | | | | | | | | |
| Participant | 1 | 1 | 1 | 1 | 1 | 1 | 1 | 1 |
| 16 | | | | | | | | |
| Participant | 2 | 2 | 2 | 1 | 2 | 2 | 1 | 2 |
| 17 | | | | | | | | |
| Participant | 3 | 4 | 4 | 3 | 2 | 2 | 3 | 2 |
| 18 | | | | | | | | |

Gas, bloating and abdominal discomfort were assessed in the GI Questionnaire using a 5 point scale, with a higher score signifying a better overall GI feeling and less dietary side effects.

For both treatments, an average baseline score of 3 (Occasionally) was answered for “How often do you experience gas?” After treatment with Invention Embodiment, the average response remained at 3. However, after treatment with Control the average score decreased to 2 (Regularly).

When participants were asked “How often do you have stomach rumbling/bloating?” an average baseline score for both treatments of 3 (Occasionally) was answered. After treatment with Invention Embodiment, the average response increased to 4 (Rarely). However, after treatment with Control the average score decreased to 2 (regularly).

Participants were also asked “How often do you have discomfort or pain anywhere in your abdomen?” An average baseline score for both treatments of 4 (Rarely) was answered. After treatment with Invention Embodiment, the average response remained at 4. However, after treatment with Control the average score decreased to 2 (Regularly).

Lastly, participants were asked “How would you describe your overall gut feeling?” An average baseline score for both treatments of 3 (Regular) was answered. After treatment with Invention Embodiment, the average response increased to 4 (Good). However, after treatment with Control (Active Control of Inulin) the average score decreased to 2 (Slightly Upset).

This study demonstrated that the ingestion of IMO fibre and probiotics according to the invention is associated with less dietary side effects in comparison to Inulin. Participants taking the novel probiotics and IMO fibre combination had no significant changes in gas and abdominal discomfort or pain. No significant changes (a decline of 2 or more points in Overall GI Questionnaire Score) were observed for the majority (89%) of participants.

Surprisingly, improvements were also noted for the embodiment of the invention in stomach rumbling/bloating and overall gut feeling.

Inulin ingestion resulted in increased gas, bloating, abdominal discomfort and pain, stomach rumbling/bloating, and decreased overall gut feeling for 67% of participants.

These results show that IMO fibre according to the present invention is well tolerated and results in less gas, bloating, and abdominal discomfort in comparison to a traditional Inulin fibre source provided in the same dosage form. In addition, this study found for the first time that the pairing of IMO fibre and probiotics (dose of 250 million CFU Bacillus coagulans IS-2) in a gummy format is well tolerated. New health benefits were also observed which may be attributed to synergistic effects of the two actives in the gummy format resulting in improvements in stomach rumbling/bloating and overall gut feeling.

All of the foregoing publications are all incorporated herein by reference in their entirety, and for the teachings contained therein.

We claim:

1. A gummy composition comprising a probiotic and isomalto-oligosaccharides.

2. A gummy composition according to claim 1 wherein the probiotic is a Bacillus strain.

3. A gummy composition according to claim 2 wherein the Bacillus strain is Bacillus coagulans strain Unique IS2.

4. A gummy composition according to claim 3 wherein the Bacillus strain is present in an amount of 250 million CFU.

5. A gummy composition according to claim 4 comprising 2.25 grams of isomalto-oligosaccharides per serving.

6. A gummy composition according to claim 5 wherein the composition provides less stomach discomfort, flatulence, stomach rumbling, belching and provides improved overall gut feeling.

7. A gummy composition according to claim 6 that tastes good.

8. A gummy composition according to claim 6 that is well tolerated.

9. A method of reducing stomach discomfort, flatulence, stomach rumbling, belching and improving overall gut feeling comprising providing a probiotic and isomalto-oligosaccharides.

10. A method of reducing stomach rumbling and bloating and improving overall gut feeling comprising providing a probiotic and isomalto-oligosaccharides.

11. A method according to claim 9 wherein the probiotic and isomalto-oligosaccharides are provided in a gummy dosage form.

12. A method according to claim 11 wherein the probiotic and isomalto-oligosaccharides are provided in a good tasting gummy dosage form.

13. A method according to claim 11 wherein the probiotic and isomalto-oligosaccharides are provided in a well-tolerated gummy dosage form.
